(19) United States
(12) Patent Application Publication
(10) Pub. No.: US 2025/0195750 A1
(43) Pub. Date: Jun. 19, 2025

(54) INFUSION DEVICE

(71) Applicants: INVENTEC APPLIANCES CORPORATION, New Taipei City (TW); INVENTEC EASY DOCTOR CORPORATION, SHANGHAI (CN)

(72) Inventors: SHIENG-TING KAO, New Taipei City (TW); YUAN-CHENG CHUANG, New Taipei City (TW)

(21) Appl. No.: 18/608,854

(22) Filed: Mar. 18, 2024

(30) Foreign Application Priority Data
Dec. 13, 2023 (TW) 112148418

(51) Int. Cl. A61M 5/142

(52) U.S. Cl. CPC A61M 5/14228; A61M 5/14232; A61M 2205/103; A61M 2205/18; A61M 2205/3306; A61M 2205/3334; A61M 2205/3365; A61M 2205/502

(57) ABSTRACT

An infusion device includes a peristaltic pump, a gear set, an optical encoder, a motor, and a control module. The peristaltic pump is used to transport a fluid in an infusion tube. The motor is used to output power to drive the peristaltic pump, such that the peristaltic pump squeezes the infusion tube to transport the fluid. The optical encoder is driven by the motor to operate synchronously with the peristaltic pump. The control module is electrically connected to the motor and the optical encoder. The optical encoder is used to detect a rotational speed of the motor. The control module detects a flow rate of the fluid in the infusion tube based on the rotational speed, and the control module controls the rotational speed to change a peristaltic frequency of the peristaltic pump squeezing the infusion tube to adjust the flow rate.

### CROSS-REFERENCE TO RELATED PATENT APPLICATION

This application claims the benefit of priority to Taiwan Patent Application No. 112148418, filed on Dec. 13, 2023. The entire content of the above identified application is incorporated herein by reference.

### FIELD OF THE DISCLOSURE

The present disclosure relates to an infusion device, and more particularly to an infusion device capable of transporting a fluid stably.

### BACKGROUND OF THE DISCLOSURE

In the medical field, an infusion device is a medical equipment that uses an infusion pump to transport specific medicinal liquid to a human body. When performing an infusion of a medicinal liquid such as an intravenous drip, the medicinal liquid needs to be supplied to the human body in a fixed quantity and at a constant speed. Once an unexpected increase, decrease or interruption in supply of the medicinal liquid occurs but cannot be automatically identified and dealt with immediately, the patient's health or life will be in danger.

In the relevant art, the medical personnel visually observes the infusion situation when operating the infusion device, and identifies whether the current volume and flow rate during the infusion are appropriate based on their own experience. However, the existing ways of operating the infusion device rely on the experience of the medical personnel, and are easily limited by insufficient medical manpower and cannot immediately identify and handle emergencies (i.e., the above-mentioned situation of the unexpected increase, decrease or interruption in supply of the medicinal liquid occurs in the infusion device). Therefore, how to improve structural design of the infusion device and overcome the above-mentioned inadequacy has become an important issue to be addressed in the relevant art.

### SUMMARY OF THE DISCLOSURE

In response to the above-referenced technical inadequacy, the present disclosure provides an infusion, which can address an issue of an unexpected increase, decrease or interruption in supply of the medicinal liquid occurs in the existing infusion device but cannot be automatically identified and dealt with immediately.

In order to solve the above-mentioned problems, one of the technical aspects adopted by the present disclosure is to provide an infusion device, which includes a peristaltic pump, a gear set, an optical encoder, a motor, and a control module.

By virtue of “the optical encoder being used to detect a rotational speed of the motor, the control module detecting a flow rate of the fluid in the infusion tube based on the rotational speed, and the control module controlling the rotational speed to change a peristaltic frequency of the peristaltic pump squeezing the infusion tube to adjust the flow rate,” the infusion device can automatically detect the flow rate of the fluid to determine whether the supply of the medicinal liquid is abnormal. When an abnormality in the supply of the medicinal liquid is detected, the infusion device can immediately and automatically adjust the frequency of the peristaltic pump squeezing the infusion tube, thereby adjusting the supply rate of the medicinal liquid to achieve a stable infusion.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a functional block diagram of an infusion device according to the present disclosure;

FIG. 2 is a schematic view of the infusion device according to the present disclosure;

FIG. 3 is a schematic view of an optical encoder according to the present disclosure;

FIG. 4 is a schematic view of a first embodiment of a grating plate according to the present disclosure;

FIG. 5 is a schematic view of a second embodiment of the grating plate according to the present disclosure;

FIG. 6 is curve diagram showing volume flow rate of a peristaltic pump according to the present disclosure;

FIG. 7 is a schematic view of a first state showing the peristaltic pump squeezing an infusion tube according to the present disclosure;

FIG. 8 is a schematic view of a second state showing the peristaltic pump squeezing the infusion tube according to the present disclosure; and

FIG. 9 is a schematic view of a third state showing the peristaltic pump squeezing the infusion tube according to the present disclosure.

### DETAILED DESCRIPTION OF THE EXEMPLARY EMBODIMENTS

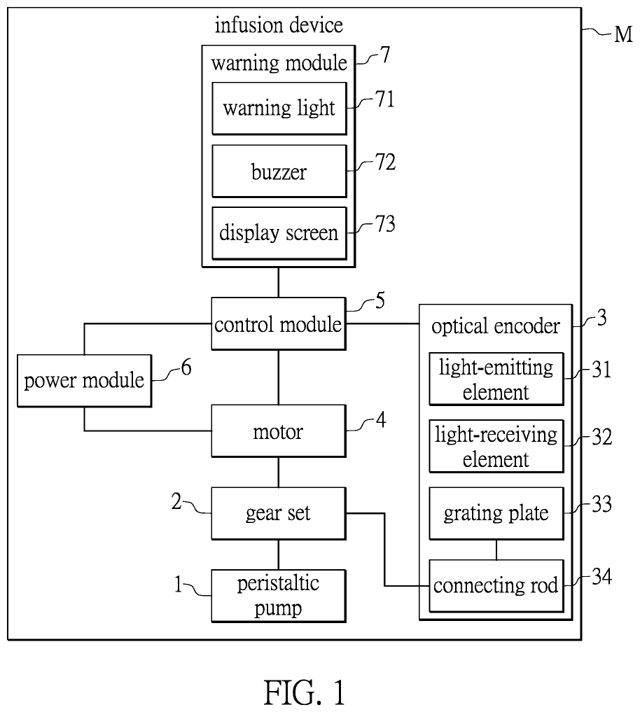

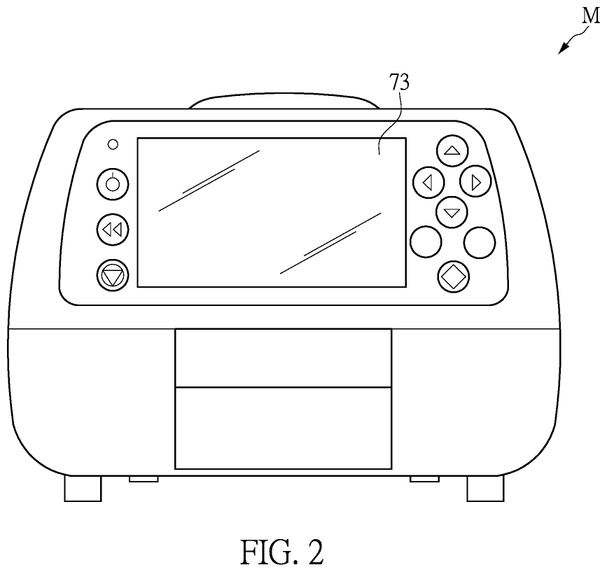

Referring to FIG. 1 and FIG. 2, FIG. 1 is a functional block diagram of an infusion device according to the present disclosure, and FIG. 2 is a schematic view of the infusion device according to the present disclosure. The present disclosure provides an infusion device M, which includes a peristaltic pump 1, a gear set 2, an optical encoder 3, a motor 4, and a control module 5.

The peristaltic pump 1 is coupled to an infusion tube P, and the peristaltic pump 1 is used to transport a fluid (not shown in the figures) in the infusion tube P. The gear set 2 is connected to the peristaltic pump 1. The optical encoder 3 is connected to the gear set 2. The motor 4 is connected to the gear set 2. In other words, the peristaltic pump 1 and the optical encoder 3 are connected to the motor 4 through the gear set 2. The motor 4 is used to output power to drive the peristaltic pump 1, such that the peristaltic pump 1 squeezes the infusion tube P to transport the fluid. The optical encoder 3 is driven by the motor 4 to operate synchronously with the peristaltic pump 1. The optical encoder 3 is used to detect a rotational speed of the motor 4. The control module 5 is electrically connected to the motor 4 and the optical encoder 3. For example, the motor 4 can be a step motor, and the control module 5 can be a microcontroller, but the present disclosure is not limited thereto.

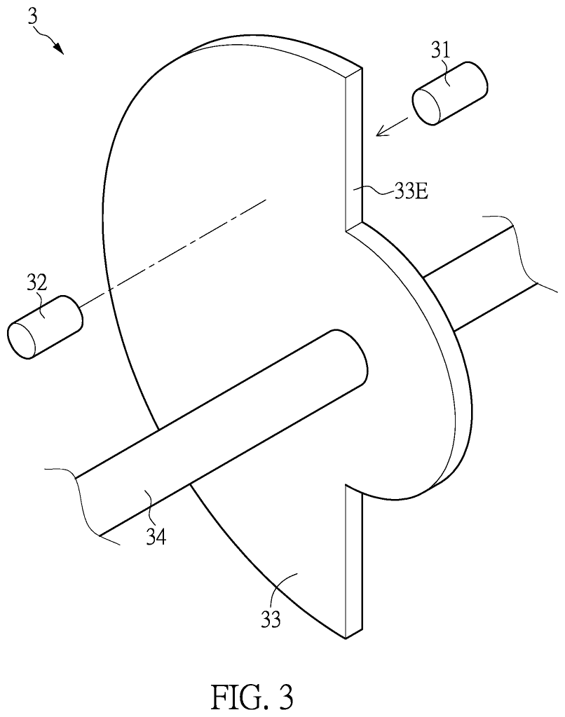

Referring to FIG. 3, FIG. 3 is a schematic view of an optical encoder according to the present disclosure. The optical encoder 3 includes at least one light-emitting element 31, at least one light-receiving element 32, a grating plate 33, and a connecting rod 34. The at least one light-emitting element 31 and the at least one light-receiving element 32 are disposed at both sides of the grating plate 33, respectively. The connecting rod 34 is connected to the grating plate 33 and the gear set 2, and the connecting rod 34 is connected to the motor 4 through the gear set 2. Therefore, the grating plate 33 is driven by the motor 4 and rotates relative to the connecting rod 34 that serves as an axis. For example, the at least one light-emitting element 31 can be a light emitting diode (LED), and the at least one light-receiving element 32 can be a photodetector. A light emitted by at least one light-emitting element 31 can be received and converted into a signal by the at least one light-receiving element 32. In addition, quantities of the light-emitting element 31 and the light-receiving element 32 are not limited in the present disclosure.

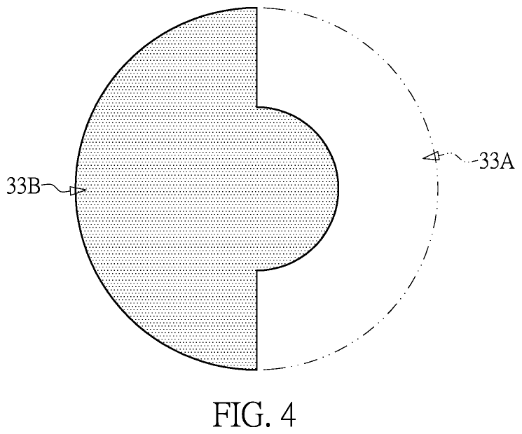

A shape of the grating plate 33 is not limited in the present disclosure. Referring to FIG. 4, FIG. 4 is a schematic view of a first embodiment of a grating plate according to the present disclosure. The grating plate 33 is driven by the motor 4 to rotate, the grating plate 33 can generate two sections, and one of the two sections can be penetrated by light but the other cannot. Specifically, a rotation range of the grating plate 33 is divided into a first part 33A which is light-permeable and a second part 33B which is opaque. As shown in FIG. 4, the at least one light-emitting element 31 and the at least one light-receiving element 32 are fixed in position, and the grating plate 33 rotates therebetween. Therefore, a part of the light emitted by the at least one light-emitting element 31 is blocked by the first part 33A of the grating plate 33, and another part of the light emitted by the at least one light-emitting element 31 passes through the second part 33B of the grating plate 33 and is received by the at least one light-receiving element 32. Then, the at least one light-receiving element 32 outputs and transmits a detection signal to the control module 5.

In the present disclosure, the grating plate 33 and the peristaltic pump 1 are driven by the motor 4 to operate synchronously. A rotational speed of the grating plate 33 is the same as a peristaltic frequency of the peristaltic pump 1, and more specifically, a period of the grating plate 33 rotating once is exactly a period for one peristaltic cycle of the peristaltic pump 1. Therefore, the optical encoder 3 detects the rotational speed of the motor 4 and feeds back the result of the detection (i.e., the detection signal) to the control module 5. The control module 5 can further detect the peristaltic frequency of the peristaltic pump 1 and a flow rate of the fluid in the infusion tube P, and determine whether the transportation of the fluid of the peristaltic pump 1 is abnormal. Furthermore, the control module 5 can output a control signal based on the rotational speed of the motor 4 detected by the optical encoder 3, so as to control the rotational speed of the motor 4 to change the peristaltic frequency of the peristaltic pump 1 squeezing the infusion tube P to adjust the flow rate in the infusion tube P.

Reference is further made to FIGS. 1 and 2. The infusion device M of the present disclosure further includes a power module 6 and a warning module 7. The warning module 7 is electrically connected to the control module 5. The power module 6 is electrically connected to the control module 5 and the motor 4. The power module 6 is used to provide power to the motor 4. For example, the warning module 7 includes a warning light 71, a buzzer 72, and a display screen 73. When the control module 5 detects the flow rate in the infusion tube P is higher than an upper limit or lower than a lower limit, the warning module 7 is used to send a warning message to the control module 5. The warning message can be a light, sound, an image, or a text. For example, the power module 6 can be a rechargeable battery. Moreover, when the control module 5 detects the flow rate in the infusion tube P is higher than the upper limit or lower than the lower limit, the control module 5 is used to control the power module 6 to turn off the power, and stop the operation of the motor 4 and the infusion of peristaltic pump 1. In addition, the infusion device M can further include a communication module (not shown in the figures) electrically connected to the control module 5. A signal connection occurs between the communication module and a remote server (not shown in the figures), such that the infusion device M can upload various information obtained during the process of the infusion to the remote server for storage.

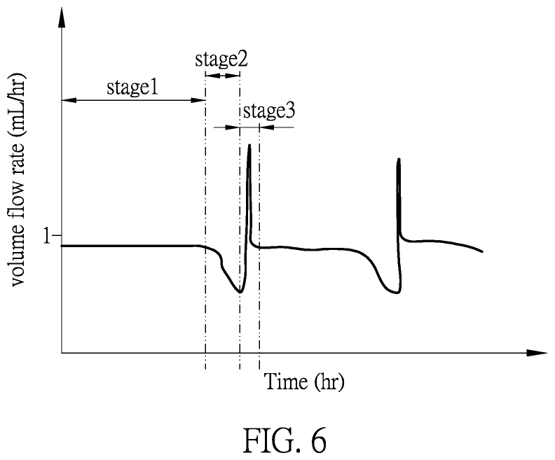

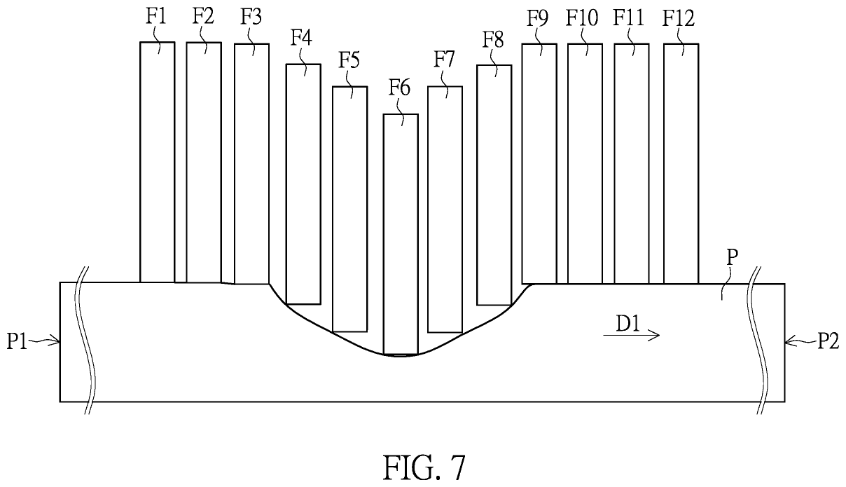

Referring to FIG. 6 to FIG. 9, FIG. 6 is curve diagram showing volume flow rate of a peristaltic pump according to the present disclosure, and FIG. 7 to FIG. 9 are schematic views of different steps showing the peristaltic pump squeezing an infusion tube according to the present disclosure. The peristaltic pump 1 is an infusion component used to pump various fluids. The specific type of peristaltic pump 1 is not limited in the present disclosure. The peristaltic pump 1 can be a linear pump, which includes a plurality of finger units F1, F2, F3, F4, F5, F6, F7, F8, F9, F10, F11, and F12 that are linearly arranged. The finger units can be finger-shaped columns. The infusion tube P can be an elastic flexible tube. When the peristaltic pump 1 pumps the fluid, the finger units F1 to F12 continuously squeeze the infusion tube P in a sequence from the finger unit F1 to the finger unit F12. Each of the finger units moves back and forth between an unpressed position and a pressed position. The finger unit squeezes the infusion tube P when moving from the unpressed position to the pressed position. A squeezed part of the infusion tube P is deformed to increase the pressure in the squeezed part of the infusion tube P, thereby pumping the fluid in the infusion tube P to flow from an inlet P1 to an outlet P2 along an infusion direction D1.

Specifically, during the stage of the finger units F1 to F7 sequentially squeezing the infusion tube P, the fluid in the infusion tube P flows at a fixed flow rate (i.e., volume flow rate), as shown in Stage 1 in FIG. 6. This Stage 1 can be further illustrated by an example of FIG. 7, which shows a stage of the finger units F4 to F8 sequentially squeezing the infusion tube P. When the infusion tube P is squeezed by the finger units F4 to F8, the squeezed part of the infusion tube P that is squeezed by the acupressure units F4 to F8 is deformed to enlarge the pressure of the infusion tube P in the squeezed part.

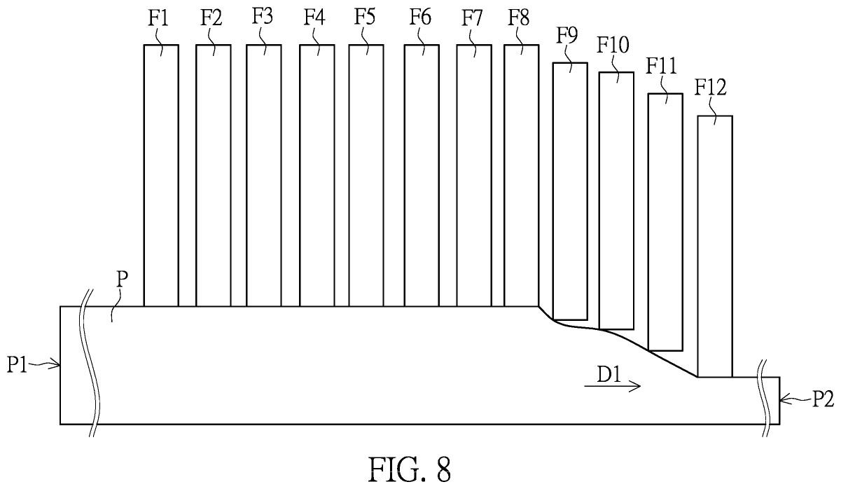

As shown in FIG. 8, the finger units F4˜F8 sequentially move back to the unpressed position, and the finger units F9˜F12 continuously move to the pressed position to squeeze the infusion tube P. The shape of the squeezed part of the infusion tube P previously squeezed by the acupressure units F4˜F8 recovers, such that the pressure in this part is reduced, and the fluid continues to be guided to flow from the inlet P1 to the outlet P2 along the infusion direction D1.

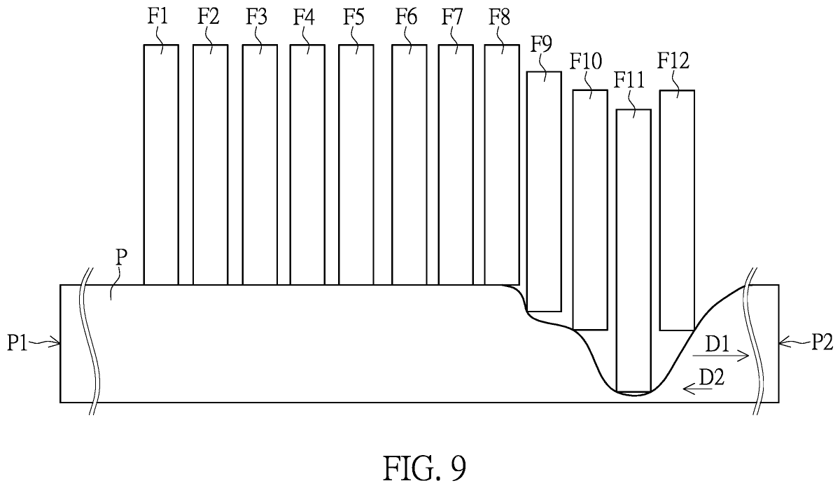

As shown in FIG. 9, when the finger unit F12 located at the end of the infusion tube P that is closest to the outlet P2 moves from the pressed position to the unpressed position, the fluid on the outlet P2 flows back along a return direction D2. At the same time, the fluid flowing along the infusion direction D1 toward the outlet P2 collides with the fluid flowing back along the return direction D2 to offset the overall flow rate, thereby decreasing the flow rate (i.e., the volume flow rate) of the fluid, which is shown in stage 2 in FIG. 6.

Reference is further made to FIG. 4 and FIGS. 6 to 9, as mentioned above, the period of the grating plate 33 rotating once is exactly the period for one peristaltic cycle of the peristaltic pump 1. The peristaltic cycle starts with the finger unit F1 squeezing the infusion tube P and ends with the finger unit F12 squeezing the infusion tube P. In order to overcome the problem that the flow rate decreases due to the backflow of the fluid in the outlet P2, the stage in which the finger units F1 to F7 squeeze the infusion tube P is set to be synchronized with the stage in which the first part 33A in the rotation range of the grating plate 33 passes through a position between the at least one light-emitting element 31 and the at least one light-receiving element 32, the stage in which the finger units F8 to F11 squeeze the infusion tube P is set to be synchronized with the stage in which the second part 33B in the rotation range of the grating plate 33 passes through the position between the at least one light-emitting element 31 and the at least one light-receiving element 32, and the stage in which the finger unit F12 squeezes the infusion tube P is set to be synchronized with the stage in which a convergence 33E (as shown in FIG. 3) between the first part 33A and the second part 33B passes through the position between the at least one light-emitting element 31 and the at least one light-receiving element 32.

In other words, while the finger units F1 to F7 are squeezing the infusion tube P, the first part 33A passes through the position between the at least one light-emitting element 31 and the at least one light-receiving element 32. While the finger units F8 to F11 are squeezing the infusion tube P, the second part 33B passes through the position between the at least one light-emitting element 31 and the at least one light-receiving element 32. While the finger unit F12 is squeezing the infusion tube P, the convergence 33E between the first part 33A and the second part 33B passes through the position between the at least one light-emitting element 31 and the at least one light-receiving element 32.

When the first part 33A passes through the position between the at least one light-emitting element 31 and the at least one light-receiving element 32, the light emitted by at least one light-emitting element 31 passes through the first 33A which is light-permeable and is received by the at least one light-receiving element 32, and the motor 4 drives the finger units F1 to F7 in the peristaltic pump 1 at a fixed rotational speed to squeeze the infusion tube P, so as to maintain the fluid in the infusion tube P to flow at a first predetermined flow rate.

When the second part 33B passes through the position between the at least one light-emitting element 31 and the at least one light-receiving element 32, the light emitted by the at least one light-emitting element 31 is blocked by the second part 33B of the grating plate 33 which is opaque and cannot be received by the at least one light-receiving element 32. At the same time, the control module 5 is used to control the motor 4 to increase the rotational speed so as to drive the finger units F8 to F11 in the peristaltic pump 1 to squeeze the infusion tube P, thereby increasing the flow rate of the fluid in the infusion tube P to a second predetermined flow rate to offset the backflow of the fluid in the outlet P2 of the infusion tube P. As shown in stage 3 in FIG. 6, in the stage 3, the fluid with increased flow rate collides with the fluid flowing back to form a pulse. Preferably, the second predetermined flow rate is 16 times the first predetermined flow rate. When the grating plate 33 rotates once and the convergence 33E between the first part 33A and the second part 33B passes through the position between the at least one light-emitting element 31 and the at least one light-receiving element 32, the control module 5 is used to control the motor 4 to reduce the rotational speed and drive the finger unit F12 in the peristaltic pump 1 to squeeze the infusion tube P, such that the fluid in the infusion tube P returns to the state of flowing at the first predetermined rate. Since the operation of the peristaltic pump 1 is a continuous process, the control module 5 can be preset to control the motor 4 to increase the rotational speed when the finger units F8 to F11 in the peristaltic pump 1 squeeze the infusion tube P, so as to increase the flow rate and offset the backflow of the fluid in the outlet P2.

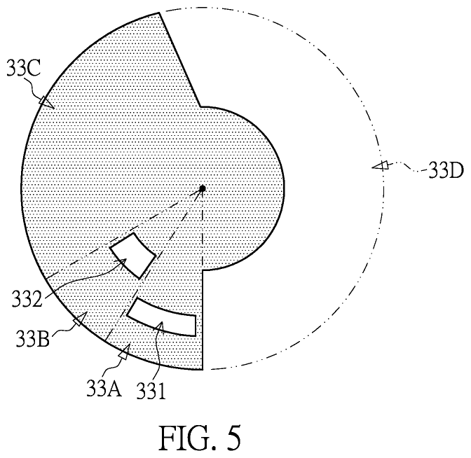

Referring to FIG. 5, FIG. 5 is a schematic view of a second embodiment of the grating plate according to the present disclosure. The grating plate 33 has a first through hole 331 and a second through hole 332, and the second through hole 332 is more adjacent to a rotational axis of the grating plate 33 than the first through hole 331. The rotation range of the grating plate 33 is divided into a first part 33A, a second part 33B, a third part 33C, and a fourth part 33D. The first through hole 331 is located at the first part 33A, the second through hole 332 is located at the second part 33B, the third part 33C is opaque, the fourth part 33D is light-permeable, the first part 33A is contiguous to the second part, and the first part 33A and the second part 33B are located between the third part 33C and the fourth part 33D.

Referring to FIGS. 5 to 9, in the second embodiment, the stage in which the finger units F1 to F6 squeeze the infusion tube P is set to be synchronized with the stage in which the fourth part 33D in the rotation range of the grating plate 33 passes through a position between the at least one light-emitting element 31 and the at least one light-receiving element 32. The stage in which the finger unit F7 squeezes the infusion tube P is set to be synchronized with the stage in which the first part 33A in the rotation range of the grating plate 33 passes through the position between the at least one light-emitting element 31 and the at least one light-receiving element 32. The stage in which the finger unit F8 squeezes the infusion tube P is set to be synchronized with the stage in which the second part 33B in the rotation range of the grating plate 33 passes through the position between the at least one light-emitting element 31 and the at least one light-receiving element 32. The stage in which the finger units F9 to F11 squeeze the infusion tube P is set to be synchronized with the stage in which the third part 33C in the rotation range of the grating plate 33 passes through the position between the at least one light-emitting element 31 and the at least one light-receiving element 32. The stage in which the finger unit F12 squeezes the infusion tube P is set to be synchronized with the stage in which a convergence between the third part 33C and the fourth part 33D in the rotation range of the grating plate 33 passes through the position between the at least one light-emitting element 31 and the at least one light-receiving element 32.

In other words, while the finger units F1 to F6 are squeezing the infusion tube P, the fourth part 33D passes through the position between the at least one light-emitting element 31 and the at least one light-receiving element 32. While the finger unit F7 is squeezing the infusion tube P, the first part 33A passes through the position between the at least one light-emitting element 31 and the at least one light-receiving element 32. While the finger unit F8 is squeezing the infusion tube P, the second part 33B passes through the position between the at least one light-emitting element 31 and the at least one light-receiving element 32. While the finger units F9 to F11 are squeezing the infusion tube P, the third part 33C passes through the position between the at least one light-emitting element 31 and the at least one light-receiving element 32. While the finger unit F12 is squeezing the infusion tube P, the convergence between the third part 33C and the fourth part 33D passes through the position between the at least one light-emitting element 31 and the at least one light-receiving element 32.

Furthermore, when the peristaltic pump 1 performs one peristaltic cycle, the grating plate 33 rotates once synchronously. When the fourth part 33D passes through the position between the at least one light-emitting element 31 and the at least one light-receiving element 32, the light emitted by the at least one light-emitting element 31 hits on the fourth part 33D which is light-permeable and is received by the at least one light-receiving element 32. At the same time, the motor 4 drives the finger units F1 to F6 in the peristaltic pump 1 at a fixed rotational speed to squeeze the infusion tube P, so as to maintain the fluid in the infusion tube P to flow at a first predetermined flow rate.

When the first part 33A passes through the position between the at least one light-emitting element 31 and the at least one light-receiving element 32, the light emitted by the at least one light-emitting element 31 hits on the first part 33A, a part of the light passes through the first through hole 331 and is received by the at least one light-receiving element 32, another part of the light is blocked by the grating plate 33, and the motor 4 drives the finger unit F7 in the peristaltic pump 1 at the fixed rotational speed to maintain the fluid in the infusion tube P to flow at the first predetermined flow rate.

When the second part 33B passes through the position between the at least one light-emitting element 31 and the at least one light-receiving element 32, the light emitted by the at least one light-emitting element 31 hits on the second part 33B, a part of the light passes through the second through hole 332 and is received by the at least one light-receiving element 32, another part of the light is blocked by the grating plate 33, and the control module 5 is used to control the motor 4 to increase the rotational speed so as to drive the finger unit F8 in the peristaltic pump 1 to squeeze the infusion tube P, thereby increasing the flow rate of the fluid in the infusion tube P to a second predetermined flow rate. The second predetermined flow rate is 16 times the first predetermined flow rate. Therefore, the effect of slowing down the flow rate caused by the backflow of the fluid in the outlet P2 of the infusion tube P can be offset. As shown in stage 3 in FIG. 6, in the stage 3, the fluid with increased flow rate collides with the fluid flowing back to form a pulse.

It should be noted that the first through hole 331 and the second through hole 332 have different positions on the grating plate 33, and the second through hole 332 is closer to the rotation axis of the grating plate 33 than the first through hole 331. Therefore, when the first part 33A and the second part 33B pass through the position between the at least one light-emitting element 31 and the at least one light-receiving element 32, the at least one light-receiving element 32 can receive different light signals.

When the third part 33C passes through the position between the at least one light-emitting element 31 and the at least one light-receiving element 32, the light emitted by the at least one light-emitting element 31 hits on the third part 33C and is blocked by the grating plate 33. At the same time, the control module 5 is used to control the motor 4 to increase the rotational speed so as to drive the finger units F9 to F11 in the peristaltic pump 1 to squeeze the infusion tube P and maintain the fluid in the infusion tube P to flow at the second predetermined flow rate. When the convergence between the third part 33C and the fourth part 33D passes through the position between the at least one light-emitting element 31 and the at least one light-receiving element 32, the control module 5 is used to control the motor 4 to reduce the rotational speed so as to drive the finger unit F12 in the peristaltic pump 1 to squeeze the infusion tube P, such that the fluid in the infusion tube P returns to the state of flowing at the first predetermined rate.

In conclusion, in the infusion device provided by the present disclosure, the optical encoder 3 detects the rotational speed of the motor 4 and feeds back the result of the detection (i.e., the detection signal) to the control module 5. The control module 5 can further detect the peristaltic frequency of the peristaltic pump 1 and a flow rate of the fluid in the infusion tube P, and determine whether the transportation of the fluid of the peristaltic pump 1 is abnormal. Furthermore, the control module 5 can control the rotational speed of the motor 4 to change the peristaltic frequency of the peristaltic pump 1 squeezing the infusion tube P based on the rotational speed of the motor 4 detected by the optical encoder 3, thereby adjusting the flow rate in the infusion tube P to achieve stable infusion. In addition, the infusion device M of the present disclosure further includes a power module 6 and a warning module 7. The power module 6 is used to provide power to the motor 4. When the control module 5 detects the flow rate in the infusion tube P is higher than an upper limit or lower than a lower limit, the warning module 7 is used to send a warning message to the control module 5, and the control module 5 is used to control the power module 6 to turn off the power, and stop the operation of the motor 4 and the infusion of peristaltic pump 1.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. An infusion device, comprising:
a peristaltic pump being used to transport a fluid in an infusion tube;
a gear set being connected to the peristaltic pump;
an optical encoder connected to the gear set;
a motor connected to the gear set, wherein the motor is used to output power to drive the peristaltic pump, such that the peristaltic pump squeezes the infusion tube to transport the fluid, and the optical encoder is driven by the motor to operate synchronously with the peristaltic pump; and
a control module electrically connected to the motor and the optical encoder, wherein the optical encoder is used to detect a rotational speed of the motor, the control module detects a flow rate of the fluid in the infusion tube based on the rotational speed, and the control module controls the rotational speed to change a peristaltic frequency of the peristaltic pump squeezing the infusion tube to adjust the flow rate.

2. The infusion device according to claim 1, wherein the optical encoder includes at least one light-emitting element, at least one light-receiving element, a grating plate, and a connecting rod, the connecting rod is connected to the grating plate and the motor, the grating plate is driven by the motor and rotates relative to the connecting rod, a rotational speed of the grating plate is same as the peristaltic frequency of the peristaltic pump, and the at least one light-emitting element and the at least one light-receiving element are respectively disposed at both sides of the grating plate.

3. The infusion device according to claim 2, wherein a rotation range of the grating plate is divided into a first part and a second part, the first part is light-permeable, and the second part is opaque; wherein, when a light emitted by the at least one light-emitting element passes through the first part and is received by the at least one light-receiving element, the motor drives the peristaltic pump at a fixed rotational speed to keep the fluid flowing in the infusion tube at a fixed flow rate; wherein, when the light emitted by the at least one light-emitting element is blocked by the second part and unable to be received by the at least one light-receiving element, the control module is used to control the rotational speed of the motor to increase the flow rate in the infusion tube.

4. The infusion device according to claim 2, wherein the grating plate has a first through hole and a second through hole, and the second through hole is more adjacent to a rotational axis of the grating plate than the first through hole.

5. The infusion device according to claim 4, wherein a rotation range of the grating plate is divided into a first part, a second part, a third part, and a fourth part, the first through hole is located at the first part, the second through hole is located at the second part, the third part is opaque, the fourth part is light-permeable, the first part is contiguous to the second part, and the first part and the second part are located between the third part and the fourth part.

6. The infusion device according to claim 5, wherein, when a light emitted by the at least one light-emitting element hits on the first part, a part of the light passes through the first through hole and is received by the at least one light-receiving element, another part of the light is blocked by the first part, and the motor drives the peristaltic pump at a fixed rotational speed to keep the fluid flowing in the infusion tube at a fixed flow rate.

7. The infusion device according to claim 5, wherein, when the light emitted by the at least one light-emitting element hits on the second part, a part of the light passes through the second through hole and is received by the at least one light-receiving element, another part of the light is blocked by the second part, and the control module is used to control the rotational speed of the motor to increase the flow rate in the infusion tube.

8. The infusion device according to claim 5, wherein, when a light emitted by the at least one light-emitting element hits on the third part, the light is blocked by the grating plate, and the control module is used to control the rotational speed of the motor to increase the flow rate in the infusion tube.

9. The infusion device according to claim 5, wherein, when a light emitted by the at least one light-emitting element hits on the fourth part, the light is received by the at least one light-receiving element, and the motor drives the peristaltic pump at a fixed rotational speed to keep the fluid flowing in the infusion tube at a fixed flow rate.

10. The infusion device according to claim 1, further comprising: a warning module and a power module, wherein the warning module includes a warning light, a buzzer, and a display screen, and the warning module is electrically connected to the control module and the motor; wherein, when the control module detects a flow rate in the infusion tube is higher than an upper limit or lower than a lower limit, the warning module is used to send a warning message to the control module; wherein, when the control module detects the flow rate in the infusion tube is higher than the upper limit or lower than the lower limit, the control module is used to control the power module to turn off the power and stop the operation of the motor.
